(19) United States
(12) Patent Application Publication
(10) Pub. No.: US 2005/0125014 A1
(43) Pub. Date: Jun. 9, 2005

(54) Gastric ring for treatment of obesity

(76) Inventors: Jean-Louis Duluco, Bordeaux (FR); Michel Therin, Lyon (FR)

Correspondence Address: OLIFF & BERRIDGE, PLC, P.O. BOX 19928, ALEXANDRIA, VA, 22320, US

(21) Appl. No.: 10/499,681

(22) PCT Filed: Jan. 8, 2003
(86) PCT No.: PCT/FR03/00039

(30) Foreign Application Priority Data
Jan. 9, 2002 (FR) 02/00262

(51) Int. Cl. A61B 17/08

(52) U.S. Cl. 606/151

(57) ABSTRACT

This gastric ring (1) comprises:
a band (3) defining a central portion (3a) which is able to surround the wall of the stomach, and two lateral portions (3b) which can be connected to one another to hold the band (3) around the stomach (2), and
means (4) for connecting said lateral portions (3b) to one another and for maintaining these lateral portions (3b) in the connected position.

According to the invention, the band (3) consists of an assembly of filaments made of a bioabsorbable material.

The present invention concerns a gastric ring for treatment of obesity. Such a ring is also presently known as a “gastroplasty ring”.

It is known to treat a patient with pathological obesity by fitting a ring round the patient's stomach in such a way as to create, in the upper part of the stomach, a pouch of small dimensions, and an opening for flow of food, also of small dimensions.

The principle of such rings is well known, and the documents WO-A-86/04498 and EP-A-0 611 561 may be cited as documents illustrating existing gastric rings.

Some of the existing rings have the disadvantage of being relatively aggressive with regard to the wall of the stomach, to the point of causing inflammation of this wall, or, in extreme cases, perforations of this wall. This aggressiveness is the result of the solid and rigid nature of this ring and in particular of the presence of an inflatable pouch situated on the internal face of the ring, this inflatable pouch making it possible to adjust the surface area of the opening delimited by the ring, by exerting on the stomach wall a pressure directed radially inward.

The implantable chambers permitting percutaneous inflation or deflation of these inflatable pouches, and the tubes connecting these chambers and these pouches, have the disadvantage of posing risks of leakage, migration and infection.

The existing rings also have the disadvantage of posing risks of tilting or sliding called “slippage” and of requiring follow-up operations when it is necessary to replace them or withdraw them after a certain period of treatment.

It is an object of the present invention to overcome all these disadvantages of the existing devices.

The ring to which the invention relates comprises in a manner known per se:
a band defining a central portion which is able to surround the wall of the stomach, and two lateral portions which are distant from one another and can be connected to one another in order to hold the band around the stomach, and
means for connecting said lateral portions to one another and for maintaining these lateral portions in the connected position.

According to the invention, the band consists of an organized or unorganized assembly of filaments or fibers made of a bioabsorbable or biodegradable material.

Such an assembly allows the band to have a deformability significantly greater than that of the rings of the prior art, which are made of a solid synthetic material with limited flexibility, and to bear in a distributed manner across the wall of the stomach. The band of the ring according to the invention is thus very much nonaggressive with regard to the wall of the patient's stomach.

Moreover, and above all, this assembly of filaments presents a porous structure allowing it to be colonized by the cells of the stomach wall until fibrosis forms around the band. This fibrosis generates scar contraction, which means there is no need to use an inflatable pouch to adjust the cross section of the opening of the ring.

The gradual tissue integration of the band secures the ring to the stomach wall and completely prevents migration (“slippage”) of the ring and produces a wide surface area of contact between tissues and band, thus promoting absorption of the latter.

This slow absorption of the filaments constituting the band permits gradual disappearance of this band, which eliminates the need for removing the ring and therefore for having to reoperate on the patient for this purpose.

The filaments or fibers can form yarns, and these yarns can be organized, for example woven or knitted. These fibers can also be assembled in a flat, unordered fashion, for example agglomerated, without weaving, in particular by intermingling and/or by means of a binder.

The filaments can be made of a lactic or polyglycolic acid polymer, or by a lactic or polyglycolic acid copolymer.

These materials have a kinetic of absorption of several months, or even several years, so as to maintain the effect of the band for a period sufficient to significantly reduce the body mass index.

Preferably, at least the face of the band intended to come into contact with the wall of the stomach preferably has a smooth coating for separating this face and this wall, at least temporarily.

This coating makes it possible to prevent or delay direct contact between the band and the stomach wall until the time when the most inflammatory phases of cicatrization of this wall have elapsed. The aforementioned tissue integration is thus separate in time from the cicatrization of the initial lesions generated by the fitting of the ring, which thus limits the risk of erosion of the stomach wall.

The porous structure of the band promotes the connection of this coating to this band.

This coating can be made of a bioabsorbable material, in particular a crosslinked collagen material.

Said connection means which the ring according to the invention comprises are preferably designed for placing said lateral portions of the band in a substantially radial direction with respect to the circle formed by the central portion of the band when the ring is placed on the stomach of a patient, these lateral portions protruding outward from this circle.

The gastric ring according to the invention does not therefore involve one of these lateral portions overlapping the other, such overlapping leading, in some devices of the prior art, to an excess thickness being created at the radially inner face of said circle, which is aggressive to the wall of the stomach.

According to a preferred embodiment of the invention, said connection means include at least one fastener comprising movable parts able to engage with said lateral portions of the band.

The knitted structure of the band makes it possible to fix said lateral portions by means of such a fastener.

Advantageously, in this case, said movable parts are displaceable between two positions, namely a position of sliding, in which the fastener can slide along said lateral portions, and a position of locking of said lateral portions, in which all possibility of sliding of the fastener relative to these lateral portions is prevented.

The sliding of the fastener along said lateral portions makes it possible to adjust the tightening of the band round the stomach.

Said lateral portions of the band can then have a length greater than that which is necessary for engagement of the fastener on these, this length being such that the fastener can be slid along these lateral portions until the desired cross section is obtained for the opening delimited by the ring, and can then be brought to the position of locking of these lateral portions.

The fastener can comprise means for immobilizing said movable parts in the aforementioned respective positions of sliding and of locking, preferably of the irreversible snap-fit type.

The fastener can be bioabsorbable or non-bioabsorbable. It can include a radiopaque substance of the barium sulfate type when it is not made of a naturally radiopaque material.

According to one possibility, the fastener comprises two movable parts which are dimensioned to surround said lateral portions of the band and to hold these portions clamped against one another, these movable parts being connected to one another at one of their longitudinal ends, by means of a film hinge.

This film hinge can be shaped in such a way as to hold said movable parts normally in a position in which one movable part is at a distance from the other, in order to facilitate engagement of the fastener on said lateral portions of the band.

One of said movable parts can comprise at least one rib protruding from its face directed toward the other movable part, this other movable part then having a slot in which the rib or ribs are intended to be engaged.

According to another possibility, the fastener is made of a non-elastically deformable material, for example a metal, and comprises deformable tabs which, in a position of deformation, are able to penetrate into the material forming said lateral portions of the band and thereby ensure fixation of these lateral portions with respect to one another.

Such a fastener made of a metal has the advantage of not taking up much space.

According to one embodiment of the band of the ring according to the invention, the band comprises a layer of monofilament yarns and three layers of multifilament yarns and is knitted on a Raschel loom with four guide bars.

The number of yarns of each of the layers depends on the desired width of the band and on the binding of each of the layers.

Thus, in the aforementioned example, for a loom comprising twenty-four one-inch needles, and for a width of band of 15 mm,
a fourth layer is formed by nine multifilament yarns;
a third layer is formed by nine monofilament yarns;
a second layer is formed by eleven multifilament yarns;
a first layer is formed by ten multifilament yarns,

and the binding of these layers is as follows:
for said fourth layer: 55/00;
for said third layer: 00/55;
for said second layer: 10/01;
for said first layer: 23/10,

(the layers being numbered according to DIN ISO 10223).

According to another embodiment of the band of the ring according to the invention, the band comprises a layer of monofilament yarns and a layer of multifilament yarns and is knitted on a Raschel loom with two needle bars and at least two guide bars; one of the guide bars is threaded with the multifilament yarns and produces stitches alternately on the front and back needles, and the other guide bar is threaded with the monofilament yarns and runs partially under three or five needles depending on the rigidity of the desired band.

In this example, the number of yarns and the binding on each of the guide bars are preferably as follows:

| | |
|---|---|
| front bar: | twenty-six multifilament yarns; |
| | binding: 1012/0121; |
| rear bar: | twenty-two monofilament yarns; |
| | binding: 5500/0055. |

The band according to one or other of the above examples has all or some of the following characteristics:
a) absence of significant longitudinal elongation, or very limited elongation (less than 10% at 5 daN), resulting from the fact that the tie between the stitches is reduced to the strict minimum in the case of a chain binding;
b) absence of curling, resulting from this chain binding and particularly from the partial weft under three or five needles with monofilament yarns;
c) flatness of the band obtained, resulting particularly from said weft with the monofilament yarns, more rigid than those used for the binding;
d) production of nonaggressive and atraumatic longitudinal edges, resulting from knitting of the band to the desired width for this band; a band obtained by cutting from a knit of greater width than this desired width of band would in fact lead to longitudinal edges which are aggressive and traumatic with regard to the wall of the stomach, resulting from the weft of monofilament yarns, even when this is done by hot-cutting;
e) nonaggressiveness of the main faces of the band, resulting from the judicious placement of the monofilament yarns and of the multifilament yarns, the fineness of the gauge and the diameter of the yarns used;
f) limited fraying in the case of cutting, resulting from the choice of binding of each of the layers;
g) optimal compromise between the porosity of the band, for deep reintegration of tissue, the low density of yarns, necessary for absorption with minimal inflammation, the resistance of the band to elongation and its atraumatic nature, resulting from the optimized ratio of multifilament yarns and monofilament yarns.

To ensure that it is clearly understood, the invention is again described below with reference to the attached diagrammatic drawing which shows, by way of nonlimiting examples, two possible embodiments of the gastric ring to which the invention relates.

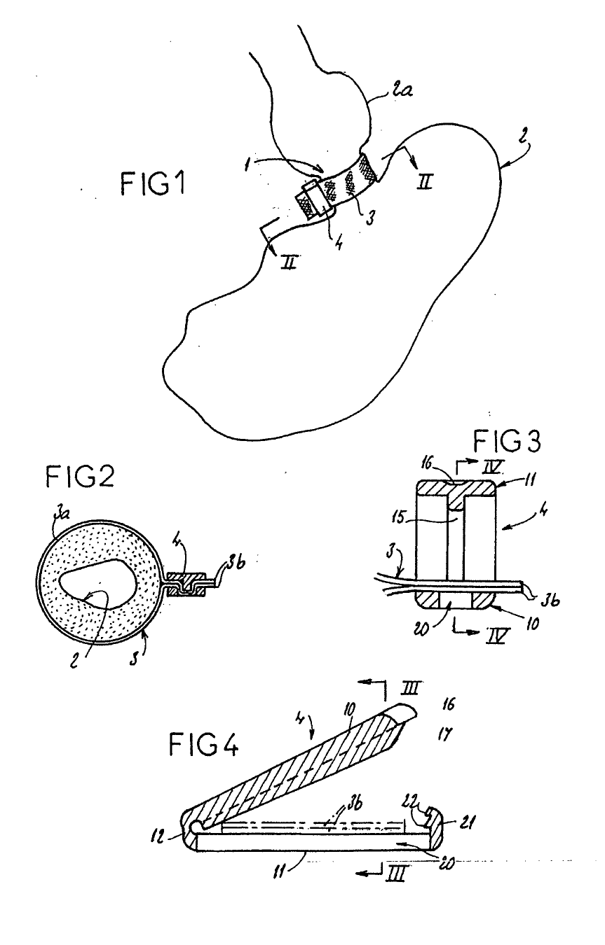

FIG. 1 is a perspective view of a gastric ring when it is in place on a patient's stomach;

FIG. 2 is a cross section thereof along the line II-II in FIG. 1;

FIG. 3 is a view, on an enlarged scale, of a fastener which it comprises, in a first embodiment and in a cross section along the line III-III in FIG. 4, and in the open position of this fastener;

FIG. 4 is a view of the fastener in cross section along the line IV-IV in FIG. 3;

FIG. 5 is a view of the fastener similar to FIG. 3, in a position of partial closure of this fastener, permitting sliding of this fastener relative to lateral portions of a band comprised by the ring;

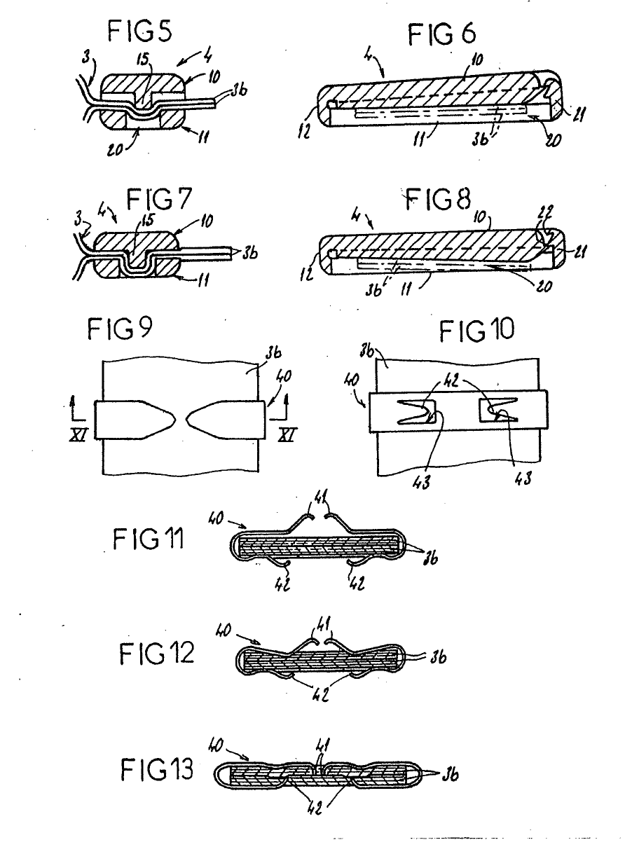

FIG. 6 is a view of the fastener similar to FIG. 4, in this same position of partial closure;

FIG. 7 is a view of the fastener similar to FIG. 3, in a position of complete closure of this fastener, preventing any possibility of the fastener sliding relative to the lateral portions of said band;

FIG. 8 is a view of the fastener similar to FIG. 4, in this same position of complete closure;

FIG. 9 is a side view of the fastener, according to a second embodiment;

FIG. 10 is a view of this fastener in a direction opposite to that according to FIG. 9;

FIG. 11 is a view of this fastener in cross section along the line XI-XI in FIG. 9, the fastener being in the open position;

FIG. 12 is a view of the fastener similar to FIG. 11, the fastener being in the partially closed position; and

FIG. 13 is a view of the fastener similar to FIG. 11, the fastener being in the completely closed position.

FIG. 1 shows a gastric ring 1 for treatment of pathological obesity of a patient, placed on the stomach 2 of this patient.

This ring 1 comprises a band 3 and a fastener 4 able to hold the band 3 around the stomach 2, in order to create, in the upper part of the stomach, a pouch 2a of small dimensions and a distal opening for flow of food, also of small dimensions.

As FIG. 2 shows, the band 3 defines a central portion 3a which is able to surround the wall of the stomach 2 in a circular trajectory, and two lateral portions 3b which can be connected to one another by means of the fastener 4, in order to hold the band 3 around the stomach 2.

The portions 3b have a length much greater than that which is necessary for the fastener 4 to engage on them. They thus permit easy manipulation of the band, and in particular its passage behind the stomach.

The band 3 is made of a knit consisting of bioabsorbable yarns, for example formed by and combining a layer of monofilament yarns and a layer of multifilament yarns.

This band 3 has been knitted for example on a Raschel loom with two needle bars and at least two guide bars, one of the guide bars being threaded with the multifilament yarns and producing stitches alternately on the front and back needles, and the other guide bar being threaded with the monofilament yarns and running partially under three or five needles depending on the rigidity of the desired band 3.

The front bar comprises twenty-six yarns, and the binding is as follows: 1012/0121; the rear bar comprises twenty-two yarns, and the binding is as folows: 5500/0055.

The basic filaments or fibers of the yarns constituting the knit are made of one or more biodegradable or bioabsorbable materials.

“Biodegradable” or “bioabsorbable” signifies the property by which a material degrades in vivo by a cellular, enzymatic or microbial mechanism (cf. for example degradation of collagen by collagenase) or by a physical-chemical mechanism (cf. for example hydrolysis of a lactic acid polymer).

Such a bioabsorbable material is preferably chosen from the group consisting of polymers of p-dioxanone, polyglycolides, polyorthoesters, polymers of trimethylene carbonate, stereocopolymers of L and D lactic acid, homopolymers of L lactic acid, copolymers of lactic acid and a compatible comonomer, such as alphahydroxy acid derivatives. Still more preferred, the bioabsorbable material has a polydispersity of less than 2.

By way of a preferred example, the biodegradable or bioabsorbable material is a lactic acid polymer (PLA) or polyglycolic acid polymer (PGA), or a copolymer of lactic acid or polyglycolic acid (PLA-PGA).

Referring to FIGS. 3 to 8, it will be seen that the fastener 4 comprises two movable parts 10, 11 which are dimensioned to surround the lateral portions 3b of the band 3 and hold these portions 3b clamped against one another in a substantially radial direction with respect to the circle which the central portion 3a forms, as is shown in FIG. 2, these lateral portions 3b protruding outward from this circle.

The movable parts 10, 11 are connected to one another via one of their longitudinal ends, by means of a film hinge 12. This film hinge 12, in the nondeformed state, holds the parts 10, 11 in the position shown in FIGS. 3 and 4, in which the part 10 is a distance away from the part 11. This spacing apart facilitates engagement of the fastener 4 on the portions 3b, particularly when this fastener 4 is put in place using minimally invasive techniques such as celioscopy or laparoscopy. The film hinge 12 can be deformed to allow the part 10 to come to the positions of partial closure and complete closure of the fastener 4, shown in FIGS. 5 and 6 and FIGS. 7 and 8, respectively.

The part 10 comprises a rib 15 protruding from its face directed toward the part 11, and a median notch 16 formed in its lateral end edge, at the end opposite from the hinge 12. In the area of the bottom of the notch 16, the rib 15 forms a snap-fit catch 17, that is to say a projection having an inclined wall at the side toward the part 11 and a plane locking wall at the side opposite from this part 11.

The part 11 has a central slot 20 in which the rib 15 is intended to be engaged, as is shown in FIGS. 7 and 8. At the end opposite from the hinge 12, this part 11 comprises a central tooth 21 which can be engaged in the notch 16 and which is equipped with two stepped snap-in catches 22. These catches 22 are able to cooperate with the catch 17, as shown respectively in FIGS. 6 and 8.

As will be appreciated with reference to FIGS. 3 to 8, these catches 17 and 22 form irreversible snap-fit means with which it is possible to lock said movable parts 10, 11 with respect to one another in two positions, namely:
a position in which the lateral portions 3b are not clamped, as is shown in FIGS. 5 and 6, in which these two portions 3b can slide with friction between the rib 15 and the portions of part 11 which longitudinally delimit the slot 20; and
a position in which these lateral portions 3b are locked, in which any possibility of sliding of these portions 3b is prevented on account of these portions 3b being clamped between the rib 15 and said portions of part 11 which longitudinally delimit the slot 20.

The fastener 4 can thus be placed on the portions 3b when it is in the open position, can be brought to the non-clamped position by simply pressing the part 10 in the direction of the part 11 so as to bring the catch 17 into engagement with the upper catch 22, can be slid along these portions 3b until the desired cross section is obtained for the admission opening for food into the stomach 2, and can be brought to the locked position by simply pressing the part 10 in the direction of the part 11 in such a way as to bring the catch 17 into engagement with the lower catch 22. The excess parts of the portions 3b can be cut off.

FIGS. 9 to 13 show a fastener 40 according to a second embodiment, formed by folding a metal band into a C shape so as to delimit a conduit in which the portions 3b can be engaged.

This fastener 40 comprises, at one end, two curved tabs 41 formed in the ends of said metal band and, at the opposite end, two curved tabs 42 individualized by suitable cutouts 43 from this same metal band. The tabs 41 are able to penetrate into one of the portions 3b, while the tabs 42 are able to penetrate into the other portion 3b.

As is shown in FIG. 12, the fastener 40 can be deformed outside the tabs 41, 42 in order to obtain a possibility of sliding of the portions 3b with friction in the fastener 40.

FIG. 13 shows that this same fastener 40 can be deformed in the area of the tabs 41, 42 so that these tabs 41, 42 penetrate into the portions 3b so as to block any possibility of sliding of these portions 3b in the fastener 40.

As will be apparent from the foregoing, the invention affords a decisive improvement to the prior art, by providing a gastric ring which is largely nonaggressive with respect to the wall of the stomach, is easy to place on the patient's stomach, and does not require removal after treatment.

A particular advantage of this ring is that it eliminates the need, after implantation, for adjusting the cross section of the opening which it delimits, hence eliminating the need for an implantable chamber.

Moreover, the tissue integration of the band, by virtue of the porous structure of the knit, generates fibrous contraction which ensures that this ring fits on the stomach.

The slow absorption of the ring allows the patients time to lose weight and to change their eating habits, while also minimizing the risks of long-term complications.

In addition, the arrangement of the band permitted by the fastener permits maximum reduction of aggressiveness when this band is in contact with the stomach wall.

1. A gastric ring for treatment of obesity, comprising:
a band defining a central portion which is able to surround the wall of the stomach, and two lateral portions which can be connected to one another to hold the band around the stomach, and
means for connecting said lateral portions to one another and for maintaining these lateral portions in the connected position;
said gastric ring being characterized in that the band consists of an assembly of filaments or fibers made of a bioabsorbable or biodegradable material.

2. The gastric ring as claimed in claim 1, characterized in that the filaments or fibers form yarns and in that these yarns are woven or knitted.

3. The gastric ring as claimed in claim 1, characterized in that fibers are agglomerated flat, without weaving or knitting, in particular by intermingling and/or by means of a binder.

4. The gastric ring as claimed in claim 1, characterized in that the filaments are made of a lactic or polyglycolic acid polymer, or by a lactic or polyglycolic acid copolymer.

5. The gastric ring as claimed in, characterized in that at least the face of the band intended to come into contact with the wall of the stomach has a smooth coating for separating this face and this wall, at least temporarily.

6. The gastric ring as claimed in claim 5, characterized in that said coating is made of a bioabsorbable material, in particular a crosslinked collagen material.

7. The gastric ring as claimed in claim 1, characterized in that said connection means are designed for placing said lateral portions of the band in a substantially radial direction with respect to the circle formed by the central portion of the band when the ring is placed on the stomach of a patient, these lateral portions protruding outward from this circle.

8. The gastric ring as claimed in claim 1, characterized in that said connection means include at least one fastener comprising movable parts able to engage with said lateral portions of the band.

9. The gastric ring as claimed in claim 8, characterized in that said movable parts are displaceable between two positions, namely a position of sliding, in which the fastener can slide along said lateral portions, and a position of locking of said lateral portions, in which all possibility of sliding of the fastener relative to these lateral portions is prevented.

10. The gastric ring as claimed in claim 9, characterized in that said lateral portions of the band have a length greater than that which is necessary for engagement of the fastener on these, this length being such that the fastener can be slid along these lateral portions until the desired cross section is obtained for the opening delimited by the ring, and can then be brought to the position of locking of these lateral portions.

11. The gastric ring as claimed in claim 8, characterized in that the fastener comprises means for immobilizing said movable parts in the respective positions of sliding and of locking.

12. The gastric ring as claimed in claim 11, characterized in that said immobilizing means are of the irreversible snap-fit type.

13. The gastric ring as claimed in claim 8, characterized in that the fastener is made of a bioabsorbable or biodegradable material.

14. The gastric ring as claimed in claim 8, characterized in that the fastener is made of a non-bioabsorbable material.

15. The gastric ring as claimed in claim 8, characterized in that the fastener comprises two movable parts which are dimensioned to surround the lateral portions of the band and to hold these portions clamped against one another, these movable parts being connected to one another at one of their longitudinal ends, by means of a film hinge.

16. The gastric ring as claimed in claim 15, characterized in that the film hinge is shaped in such a way as to hold said movable parts normally in a position in which one movable part is at a distance from the other.

17. The gastric ring as claimed in claim 15, characterized in that one of said movable parts comprises at least one rib protruding from its face directed toward the other movable part, and in that this other movable part has a slot in which the rib or ribs are intended to be engaged.

18. The gastric ring as claimed in claim 8, characterized in that the fastener is made of a non-elastically deformable material, for example a metal, and in that it comprises deformable tabs which, in a position of deformation, are able to penetrate into the knit forming said lateral portions of the band and thereby ensure fixation of these lateral portions with respect to one another.

19. The gastric ring as claimed in claim 1, characterized in that said band comprises a layer of monofilament yarns and three layers of multifilament yarns, and in that it is obtained by knitting on a Raschel loom with four guide bars.

20. The gastric ring as claimed in claim 19, characterized in that said band is obtained by knitting of said layer of monofilament yarns and said layers of multifilament yarns, and in that this band comprises, for a width of band of 15 mm,
a fourth layer formed by nine multifilament yarns;
a third layer formed by nine monofilament yarns;
a second layer formed by eleven multifilament yarns;
a first layer formed by ten multifilament yarns,
and in that the binding of these layers is as follows:
for said fourth layer: 55/00;
for said third layer: 00/55;
for said second layer: 10/01;
for said first layer: 23/10,
the layers being numbered according to DIN ISO 10223

21. The gastric ring as claimed in claim 1, characterized in that said band comprises a layer of monofilament yarns and a layer of multifilament yarns and is obtained by knitting on a Raschel loom with two needle bars and at least two guide bars, one of the guide bars being threaded with the multifilament yarns and producing stitches alternately on the front and back needles, and the other guide bar being threaded with the monofilament yarns and running partially under three or five needles depending on the rigidity of the desired band.

22. The gastric ring as claimed in claim 21, characterized in that the number of yarns in said layer of multifilament yarns is 26 multifilament yarns, and in that these yarns are placed on the front bar, with a binding 1012/0121, while the number of yarns in the layer of monofilament yarns is twenty-two monofilament yarns, engaged on the rear bar, the binding being 5500/0055.
